(19) United States
(12) Patent Application Publication
(10) Pub. No.: US 2022/0291235 A1
(43) Pub. Date: Sep. 15, 2022

(54) ACOUSTO-THERMAL SHIFT ASSAY FOR LABEL-FREE PROTEIN ANALYSIS

(71) Applicant: THE REGENTS OF THE UNIVERSITY OF COLORADO - A BODY CORPORATE, Denver, CO (US)

(72) Inventor: Xiaoyun Ding, Denver, CO (US)

(21) Appl. No.: 17/635,632

(22) PCT Filed: Aug. 28, 2020
(86) PCT No.: PCT/US20/48451

§ 371 Date: Feb. 15, 2022

Related U.S. Application Data
(60) Provisional application No. 62/894,423, filed on Aug. 30, 2019.

(51) Int. Cl. G01N 33/68; B01L 3/00

(52) U.S. Cl. CPC G01N 33/6893; B01L 3/502761; B01L 2400/0436; B01L 2400/0439; B01L 2300/0645; G01N 2800/22

(57) ABSTRACT

Thermal shift assays (TSAs) have been extensively used to study thermodynamics of proteins and provide an efficient means to assess protein-ligand binding or protein-protein interaction. However, existing TSAs have limitations such as time consuming, labor intensive, or low sensitivity. Here, an acousto-thermal shift assay (A-TSA) is disclosed and is believed to be the first ultrasound enabled TSA which can provide a real-time analysis of protein thermodynamic stability. A-TSA couples unique acoustic mechanisms to achieve protein unfolding, concentration, and measurement on a single microfluidic chip within minutes. Compared to conventional TSA methods, A-TSA provides an ultra-fast (at least 30 times faster), highly sensitive (7-34 folds higher), and label -free monitoring of protein-ligand interactions and protein stability finding applications for protein analysis in biology, medicine and fast diagnosis.

### FIELD OF THE INVENTION

The present invention is related to the field of analytical chemistry. In particular, the invention defines a new and improved device and method to detect precise differences in protein secondary, tertiary or quaternary structures. By applying standing acoustic waves to a proteinaceous fluid, differences in protein unfolding characteristics (e.g., melting temperatures) are detected which are beyond the capabilities of conventional thermal shift assays. Such improvements are based upon concomitant protein aggregation that increases local protein concentrations permitting increased temperature shift detection sensitivity.

### BACKGROUND

Proteins are essential components of organisms and participate in most biological processes. Studying protein dynamics and its interaction with other molecules impact almost every field from fundamental biology to clinical applications.

However, conventional measurement assays and sample preparation takes from many hours up to a whole day and/or requires bulky/expensive facility and experiences. This limitation is one of the main challenges that lead to high cost and long development cycles for therapeutics and drug development in pharmaceutical industry and particularly in the development of portable medical devices for fast and low cost diagnosis and healthcare.

What is needed in the art is a device and method providing a single step that rapidly and accurately identify protein characteristics for “point of care” analysis.

### SUMMARY OF THE INVENTION

The present invention is related to the field of analytical chemistry. In particular, the invention defines a new and improved device and method to detect precise differences in protein secondary, tertiary or quaternary structures. By applying standing acoustic waves to a proteinaceous fluid, differences in protein unfolding characteristics (e.g., melting temperatures) are detected which are beyond the capabilities of conventional thermal shift assays. Such improvements are based upon concomitant protein aggregation that increases local protein concentrations permitting increased temperature shift detection sensitivity.

In one embodiment, the present invention contemplates a method, comprising: a) providing: i) an acousto-thermal device comprising a surface acoustic wave source and at least one microfluidic channel or chamber; and ii) a sample comprising at least one protein; b) introducing said sample into said at least one microfluidic channel or chamber; c) controlling the temperature of the said sample with said surface acoustic wave source to a plurality of precise temperatures within said microfluidic channel or chamber under conditions that create a precipitated protein; and d) aggregating said precipitated protein with said surface acoustic wave into a pattern. In one embodiment, said pattern comprises parallel lines or arrays. In one embodiment, said aggregating increases a local concentration of said precipitated protein. In one embodiment, said aggregating is performed simultaneously with said protein precipitation. In one embodiment, the method further comprises measuring protein gray intensity. In one embodiment, said protein gray intensity measurements determine a protein melting curve. In one embodiment, said sample comprises plurality of biological cells. In one embodiment, the method further comprises lysing at least a portion of said plurality of biological cells with said surface acoustic wave source. In one embodiment, the acousto-thermal device further comprises a piezoelectric substrate comprising at least one microchannel or chamber. In one embodiment, the acousto-thermal device further comprises at least two parallel interdigital transducers deposited longitudinally in said at least one microchannel or chamber. In one embodiment, the acousto-thermal device further comprises a fluid comprising a plurality of proteins disposed between said at least two parallel interdigital transducers. In one embodiment, each of said interdigital transducer comprises thirty (30) pairs of electrodes. In one embodiment, said electrode pairs comprise chromium and gold. In one embodiment, each of said electrode pairs have a thickness of approximately 5/100 nm. In one embodiment, wherein each of said electrode pairs comprise an electrode finger of 50 μm in length, a pitch of 100 μm, and an aperture of 10 mm. In one embodiment, said electrode pairs yield a standing acoustic wave having a frequency of approximately 20 MHz. In one embodiment, said piezoelectric substrate comprises a material selected from the group consisting of silicon, glass, plastic, quartz and polydimethylsiloxane (PDMS). In some embodiments, the method comprises an acoustic microfluidic device that can control protein precipitation. In other embodiments, the method comprises distinguishing the protein solubility difference upon a protein interaction with other molecules or the solubility change due to the protein configuration change. In other embodiments, the method measurements can be done on a microchip within a few minutes without peripheral systems. In some embodiments, the method comprises controlling, aggregating and characterizing a precipitate on a single microchip without any additional systems or steps. In some embodiments, the methods comprise a low cost and fast drug screening. In other embodiments, the method comprises a fast label-free diagnostic device to diagnose diseases including, but not limited to, sickle cell disease, malaria, hemoglobinopathies or many other diseases that is related to a protein disorder where modified proteins have a melting temperature shift. In other embodiments, the method comprises at least one microfluidic channel and/or chamber, or two or more chambers or channels to simultaneously measure multiple protein samples. In other embodiments, the method comprises protein aggregation, patterning and concentrating precipitated protein. In other embodiments, the method comprises measuring either gray intensity or fluorescence intensity. In other embodiments, the method comprises cell lysis before such protein aggregation, patterning and concentrating. In other embodiments, the method comprises using surface acoustic waves for protein precipitation, protein patterning and concentration. In other embodiments, the method comprises cell lysis, protein precipitation and protein aggregation or patterning. In other embodiments the method comprises simultaneous or almost simultaneous lysis, precipitation, and aggregation or patterning. In other embodiments, the method does not comprise cell lysis.

In one embodiment, the present invention contemplates an acousto-thermal device, comprising: i) a piezoelectric substrate comprising at least one microchannel or chamber; ii) at least two parallel interdigital transducers deposited longitudinally in said at least one microchannel or chamber; and iii) a fluid comprising a plurality of proteins disposed between said at least two parallel interdigital transducers. In one embodiment, each of said interdigital transducer comprises thirty (30) pairs of electrodes. In one embodiment, said electrode pairs comprise chromium and gold. In one embodiment, each of said electrode pairs have a thickness of approximately 5/100 nm. In one embodiment, wherein each of said electrode pairs comprise an electrode finger of 50 μm in length, a pitch of 100 μm, and an aperture of 10 mm. In one embodiment, said electrode pairs yield a standing acoustic wave having a frequency of approximately 20 MHz. In one embodiment, said piezoelectric substrate comprises a material selected from the group consisting of silicon, glass, plastic, quartz and polydimethylsiloxane (PDMS).

In one embodiment, the present invention contemplates a method, comprising: a) providing: i) an acousto-thermal device comprising a surface acoustic wave source and at least two microfluidic channels or chambers; ii) a first sample comprising at least one first protein disposed in a first microfluidic channel or chamber; and iii) a second sample comprising at least one second protein disposed in a second microfluidic channel or chamber;; b) controlling the temperature of said first and second sample with said surface acoustic wave source to a plurality of precise temperatures within said microfluidic channel or chamber under conditions that create a first and second precipitated protein; c) aggregating said first and second precipitated protein with said surface acoustic wave into a first and second pattern; d) measuring a gray intensity of said first and second precipitated protein; e) determining a first and second melting temperature of said first and second precipitated protein; and f) calculating a difference between said first and second melting temperature with a three to thirty-five-fold increased sensitivity as compared to conventional thermal shift assays. In one embodiment, said second protein is bound to a ligand. In one embodiment, the ligand is selected from the group consisting of a small organic molecule, an antibody and a protein. In one embodiment, the second protein comprises a mutation as compared to a wild type sequence. In one embodiment, said difference diagnoses a genetic disease. In one embodiment, said pattern comprises parallel lines or arrays. In one embodiment, said aggregating increases a local concentration of said precipitated protein. In one embodiment, said aggregating is performed simultaneously with said protein precipitation. In one embodiment, said sample comprises plurality of biological cells. In one embodiment, the method further comprises lysing at least a portion of said plurality of biological cells with said surface acoustic wave source. In one embodiment, the acousto-thermal device further comprises a piezoelectric substrate comprising at least one microchannel or chamber. In one embodiment, the acousto-thermal device further comprises at least two parallel interdigital transducers deposited longitudinally in said at least one microchannel or chamber. In one embodiment, the acousto-thermal device further comprises a fluid comprising a plurality of proteins disposed between said at least two parallel interdigital transducers. In one embodiment, each of said interdigital transducer comprises thirty (30) pairs of electrodes. In one embodiment, said electrode pairs comprise chromium and gold. In one embodiment, each of said electrode pairs have a thickness of approximately 5/100 nm. In one embodiment, wherein each of said electrode pairs comprise an electrode finger of 50 μm in length, a pitch of 100 μm, and an aperture of 10 mm. In one embodiment, said electrode pairs yield a standing acoustic wave having a frequency of approximately 20 MHz. In one embodiment, said piezoelectric substrate comprises a material selected from the group consisting of silicon, glass, plastic, quartz and polydimethylsiloxane (PDMS). In some embodiments, the method comprises an acoustic microfluidic device that can control protein precipitation. In other embodiments, the method comprises distinguishing the protein solubility difference upon a protein interaction with other molecules or the solubility change due to the protein configuration change. In other embodiments, the method measurements can be done on a microchip within a few minutes without peripheral systems. In some embodiments, the method comprises controlling, aggregating and characterizing a precipitate on a single microchip without any additional systems or steps. In some embodiments, the methods comprise a low cost and fast drug screening. In other embodiments, the method comprises a fast label-free diagnostic device to diagnose diseases including, but not limited to, sickle cell disease, malaria, hemoglobinopathies or many other diseases that is related to a protein disorder where modified proteins have a melting temperature shift. In other embodiments, the method comprises at least one microfluidic channel and/or chamber, or two or more chambers or channels to simultaneously measure multiple protein samples. In other embodiments, the method comprises protein aggregation, patterning and concentrating precipitated protein. In other embodiments, the method comprises measuring either gray intensity or fluorescence intensity. In other embodiments, the method comprises cell lysis before such protein aggregation, patterning and concentrating. In other embodiments, the method comprises using surface acoustic waves for protein precipitation, protein patterning and concentration. In other embodiments, the method comprises cell lysis, protein precipitation and protein aggregation or patterning. In other embodiments the method comprises simultaneous or almost simultaneous lysis, precipitation, and aggregation or patterning. In other embodiments, the method does not comprise cell lysis.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” or “approximately” as used herein, in the context of any of any assay measurements refers to +/−5% of a given measurement.

The term “surface acoustic wave source” or “surface acoustic wave generator” as used herein refers to a component of a device that emits a standing acoustic wave over the surface of a fluid. For example, a surface acoustic wave source/generator comprises a microchip having a pair of interdigitated transducers in parallel.

The term “interdigitated transducer” (IDT) as used herein, refers to two interlocking comb-shaped arrays of metallic electrodes that form pairs in the fashion of a zipper. These metallic electrodes are deposited on the surface of a piezoelectric substrate, such as quartz or lithium niobate, to form a periodic structure. IDTs primary function is to convert electric signals to surface acoustic waves (SAW) by generating periodically distributed mechanical forces via piezoelectric effect (an input transducer).

The term “microfluidic” as used herein relates to components where moving fluid is constrained in or directed through one or more channels wherein one or more dimensions are 1 mm or smaller (microscale). Microfluidic channels may be larger than microscale in one or more directions, though the channel(s) will be on the microscale in at least one direction. In some instances the geometry of a microfluidic channel may be configured to control the fluid flow rate through the channel (e.g. increase channel height to reduce shear). Microfluidic channels can be formed of various geometries to facilitate a wide range of flow rates through the channels.

The term “microfluidic device” refers to a substrate comprising at least one channel that is configured to support fluid flow. Such a device may be constructed out of a variety of materials including, but not limited to, silicon, quartz, glass, plastic and/or polydimethylsiloxane (PDMS) or other polymer(s). For example, some microfluidic devices may comprise a microchip.

The term “microchannels” refer to pathways (whether straight, curved, single, multiple, in a network, etc.) through a medium (e.g., silicon, glass, polymer, etc.) that allow for movement of liquids and gasses. Channels thus can connect other components, i.e., keep components “in communication” and more particularly, “in fluidic communication” and still more particularly, “in liquid communication.” Microchannels are channels with dimensions less than 1 millimeter and greater than 1 micron. It is not intended that the present invention be limited to only certain microchannel geometries. In one embodiment, a four-sided microchannel is contemplated. In another embodiment, the microchannel is circular (in the manner of a tube) with curved walls. In yet another embodiment, a combination of circular or straight walls is used.

The term “chamber” or “microfluidic chamber” refers to an enlarged section of a microfludic channel with a volume sufficient to allow mixing of various reagents and biological samples. A microfluidic chamber may also have windows or ports to permit analytical sampling or non-invasive data collection. A microfluidic chamber may also have an inlet microchannel and an outlet channel to permit continuous flow through the microfluidic chamber for serial data collection.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The term “ligand” as used herein, refers to any compound capable of interacting with (i.e., for example, attaching, binding etc) to a binding partner under conditions such that the binding partner alters its conformational shape. For example, if a binding partner is a protein, a conformation shape change may include, but is not limited to, changes in secondary, tertiary or quaternary structure. Ligands may include, but are not limited to, small organic molecules, antibodies, and proteins/peptides.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “protein” as used herein, refers to any of numerous naturally occurring extremely complex substances (as an enzyme or antibody) that consist of amino acid residues joined by peptide bonds, contain the elements carbon, hydrogen, nitrogen, oxygen, usually sulfur. In general, a protein comprises amino acids having an order of magnitude within the hundreds. A protein may have a conformation shape described, in part, by secondary structure (e.g., twists), tertiary structure (e.g., turns) and quaternary structure (e.g., induced by binding with other proteins). When a protein binds to a ligand, changes in conformation shape may occur including changes in secondary, tertiary, quaternary structure or any combination thereof.

The term, “purified” or “isolated”, as used herein, may refer to a peptide composition that has been subjected to treatment (i.e., for example, fractionation) to remove various other components, and which composition substantially retains its expressed biological activity.

Where the term “substantially purified” is used, this designation will refer to a composition in which the protein or peptide forms the major component of the composition, such as constituting about 50%, about 60%, about 70%, about 80%, about 90%, about 95% or more of the composition (i.e., for example, weight/weight and/or weight/volume). The term “purified to homogeneity” is used to include compositions that have been purified to ‘apparent homogeneity” such that there is single protein species (i.e., for example, based upon SDS-PAGE or HPLC analysis). A purified composition is not intended to mean that all trace impurities have been removed.

As used herein, the term “substantially purified” refers to molecules, either nucleic or amino acid sequences, that are removed from their natural environment, isolated or separated, and are at least 60% free, preferably 75% free, and more preferably 90% free from other components with which they are naturally associated. An “isolated polynucleotide” is therefore a substantially purified polynucleotide.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “sample” or “biopsy” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables). For example, a pulmonary sample may be collected by bronchoalveolar lavage (BAL) which comprises fluid and cells derived from lung tissues. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The term “bind” as used herein, includes any physical attachment or close association, which may be permanent or temporary. Generally, an interaction of hydrogen bonding, hydrophobic forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

The term, “device” as used herein, describes components including, substrates, surfaces and points of contact between reagents.

The term, “substrate” as used herein, describes a material having a surface as well as solid phases which may comprise the array, microarray or microdevice. In some cases, the substrate is solid and may comprise PDMS.

The term “luminescence” and/or “fluorescence”, as used herein, refers to any process of emitting electromagnetic radiation (light) from an object, chemical and/or compound. Luminescence results from a system which is “relaxing” from an excited state to a lower state with a corresponding release of energy in the form of a photon. These states can be electronic, vibronic, rotational, or any combination of the three. The transition responsible for luminescence can be stimulated through the release of energy stored in the system chemically or added to the system from an external source. The external source of energy can be of a variety of types including chemical, thermal, electrical, magnetic, electromagnetic, physical or any other type capable of causing a system to be excited into a state higher than the ground state. For example, a system can be excited by absorbing a photon of light, by being placed in an electrical field, or through a chemical oxidation-reduction reaction. The energy of the photons emitted during luminescence can be in a range from low-energy microwave radiation to high-energy x-ray radiation. Typically, luminescence refers to photons in the range from UV to IR radiation.

The term “piezoelectric” as used herein, refers to an ability of certain crystalline materials to generate an electric charge in response to applied mechanical stress.

### BRIEF DESCRIPTION OF THE FIGURES

FIG. 1 presents a representative schematic of an Acousto-Thermal Shift Assay for fast, label-free, and low-cost protein analysis. All processes, from sample preparation to data collection and readout, can be done within a microchip connected to a smart phone.

FIG. 2 presents exemplary data showing a comparison of a palmatine thermal shift assay before and after binding to hemoglobin (Hb).
FIG. 2A: An acousto-thermal assay device (A-TSA) of the present invention.
FIG. 2B: A conventional fluorescence thermal shift assay device (C-TSA).

FIG. 3 presents exemplary data showing an improved sensitivity of an acousto-thermal shift assays (A-TSA) as compared to a conventional fluorescent thermal shift assay (C-TSA). Palmitine bound to hemoglobin (PA+Hb); Citrate synthase bound to oxaloacetate (CS+OOA).

FIG. 4 presents one embodiment of a working acousto-thermal shift assay (ATSA).
FIG. 4A(i) presents a schematic illustration of a standing SAW formed in between two IDTs within a microchannel.
FIG. 4A(ii) presents a schematic illustration protein unfolding and precipitation induced by SAW acoustic heating within a microfluidic channel
FIG. 4A(iii) presents a schematic illustration of a precipitated proteins that were aggregated and concentrated along nodes and/or antinodes of a standing acoustic field.
FIG. 4A(iv) presents exemplary data showing acoustic melting curves generated by analyzing the gray intensity of precipitated and assembled protein as a function of SAW time, where the melting time (Tₘ) was determined as the time point at which the 50% change of gray intensity (ΔI) occurs, i.e. Iₘ=(Iₘₐₓ+Iₘᵢₙ)/2.
FIG. 4B shows a representative image of an A-TSA device fabricated by bonding a PDMS substrate and a lithium niobate wafer with a pair of IDTs.
FIG. 4C presents exemplary data showing acoustic-driven hemoglobin (Hb) protein unfolding, precipitation, and assembly as demonstrated by optical images of purified protein before and after SAW actuation. Scale bars: 200 μm
FIG. 4D presents exemplary data showing acoustic-driven blood plasma mixed protein unfolding, precipitation, and assembly as demonstrated by optical images of purified protein before and after SAW actuation. Scale bars: 200 μm

FIG. 5 presents exemplary data showing that the A-TSA provides a rapid and sensitive assessment of protein-ligand binding and protein stability for two purified proteins, Hb and CS, in the absence or presence of their corresponding binding ligands, i.e. palmatine chloride (Pal) and oxaloacetic acid (OAA) as compared to conventional C-TSA methods. The arrows indicate the melting time at which the 50% change of gray intensity (Iₘ=(Iₘₐₓ+Iₘᵢₙ)/2) occurs for each curve. The Hb concentration of 31 μM and the CS concentration of 15 μM were used in these tests. All error bars represent standard deviation (s.d.). In B, D and E: NA indicates the data of thermal shifts between Hb and Hb-Pal complexes is not available for DSF-TSA. In E, ### p<0.001 versus ATSA method.
FIG. 5A presents exemplary A-TSA data of representative melting curves (Tₘ) and melting time shifts (Δtₘ) (n=4).
FIG. 5B presents exemplary SYPRO differential scanning fluorimetry (DSF) assay) data of representative melting curves (Tₘ) and melting time shifts (Δtₘ) (n=4).
FIG. 5C presents exemplary bicinchoninic acid (BCA)data of representative melting curves (Tₘ) and melting time shifts (Δtₘ) (n=3).
FIG. 5D presents exemplary data showing a relative shift (Δtₘ/tₘ₀ or ΔTₘ/Tₘ₀) of proteins upon ligand binding as a function of ligand concentration. The dashed lines represented linear regression curve fit of the data and their tangential slope was defined as sensitivity (mM⁻¹) of TSAs.
FIG. 5E presents exemplary data showing that the presently disclosed A-TSA has superior or comparable precision as compared to C-TSAs (DSF-TSA or BCA-TSA). Precision is characterized by the fold differences between the detected shift and standard deviations (s.d.) of melting time or temperature, i.e. (Δtₘ s.d. or ΔTₘ s.d.).

FIG. 6 presents exemplary data showing the effects of protein concentration on melting time tm of Hb and its shift ≠tm upon binding of Pal under SAW actuation (19.6 MHz, 3 Watt). All error bars represent standard deviation (s.d.). *** p<0.001. ns: no significant difference (p>0.05).
FIG. 6A: Representative melting curves for various Hb concentrations.
FIG. 6B:, An analysis of melting time tₘ (n=4) of Hb at various concentrations (3.875 μM-124 μM).
FIG. 6C: Representative melting curves for Pal binding to various Hb concentrations.
FIG. 6D: An analysis of melting time shift Δtₘ (n=4) of Hb and Hb-Pal complexes at various Hb concentrations (3.875 μM, 15.5 μM, and 62 μM). The change of gray intensity, melting time tm, and shift Δtₘ are not detectable at very low Hb concentration (e.g., 3.875 μM).

FIG. 7 presents exemplary data showing that the presently disclosed A-TSA demonstrates superior sensitivity and reproducibility as compared to a heat-only condition. All error bars represent standard deviation (s.d.). *** p<0.001. n=4.
FIG. 7A: Melting curves and melting time tₘ of Hb and Hb-Pal complexes were compared between a heat-only condition and A-TSA. The A-TSA microfluidic device with protein solution was placed on a transparent heating plate and the temperature of heating plate was ramped from room temperature to 75° C. Protein unfolding and protein precipitation was digitally monitored by measures of gray intensity change of proteins as a function of heating time.
FIGS. 7B-D. Thermal shift analysis by A-TSA produces 3-fold larger melting time shift (Δtm), 5-fold larger relative shift, and 5-fold higher precision than the method with heating only.

FIG. 8 presents exemplary data showing the magnitudes of melting time shift Δtₘ are tunable by adjusting SAW power in A-TSA. All error bars represent standard deviation (s.d.). *** p<0.001.
FIG. 8A: Representative melting curves.
FIG. 8B: An analysis of melting time shift Δtm (n=4) of Hb and Hb-Pal complexes under various SAW power, 3 Watt, 2.5 Watt, and 2 Watt. The magnitude of melting time shift Δtm was remarkably increased by decreasing the SAW power.
FIG. 8C: The relative shift seems not to be dependent on the SAW power.
FIG. 8D: Acoustic heating effects are sensitive to the SAW power, i.e. lower SAW power resulted in slower temperature increase (n=3), which is responsible for this tunability.

FIG. 9 presents exemplary data showing that the presently disclosed A-TSA provides sensitive detection of thermal shifts to differentiate between healthy and sickled red blood cell (RBC) lysates, providing a new point-of-care platform for diagnosis of sickle cell disease (SCD). All error bars represent standard deviation (s.d.). In a, * p<0.05. In b, ns means no significant difference (p>0.05). Scale bars: 200 μm.
FIG. 9A: A-TSA detected differences in protein stability between healthy and sickled RBC lysates (SCD) as shown by representative melting curves and analysis of melting time tₘ (n=4) or melting temperature Tₘ (n=3).
FIG. 9B: C-TSA (e.g., BCA) does not detect differences in protein stability between healthy and sickled RBC lysates (SCD), as shown by representative melting curves and analysis of melting time tₘ (n=4) or melting temperature Tₘ (n=3).
FIG. 9C: Optical images of patterned protein microfibers of healthy and sickled RBC lysates showed similar morphology.

### DETAILED DESCRIPTION OF THE INVENTION

The present invention is related to the field of analytical chemistry. In particular, the invention defines a new and improved device and method to detect precise differences in protein secondary, tertiary or quaternary structures. By applying standing acoustic waves to a proteinaceous fluid, differences in protein unfolding characteristics (e.g., melting temperatures) are detected which are beyond the capabilities of conventional thermal shift assays. Such improvements are based upon concomitant protein aggregation that increases local protein concentrations permitting increased temperature shift detection sensitivity.

In one embodiment, the present invention contemplates an on-chip acousto=thermal shift assay (A-TSA) that represents a novel thermal shift assay strategy and demonstrates unprecedented speed and sensitivity for label-free analysis of protein thermodynamic stability in real time. A-TSA requires less sample volume and is faster than any conventional TSA in measuring single protein melting curve without any needs of molecular markers, allowing its broad potential applications in fast diagnosis. Data presented herein demonstrates a superior sensitivity to detect protein stability than conventional fluorescence-based TSA methods. Consequently, A-TSA has a superior advantage over all C-TSAs in having an improved quantitative protein measurement and precise binding affinity. A-TSA is also highly compatible with automatic processing techniques and cellular phone interface. A-TSA is able to benefit a plethora of applications in fundamental biomedical research, drug industry and fast diagnosis.

Although it is not necessary to understand the mechanism of an invention it is believed that although surface acoustic waves can pattern microparticles and cells, that ability does not necessarily lead to protein patterning. It is further believed that because protein precipitation and patterning involves small molecule aggregation, it is not obvious in comparison to SAW aggregation of larger objects such as biological cells or microbeads. Notably, the present invention makes the first disclosure of the ability of SAW to generate precipitated protein patterns subsequent to protein aggregation and concentration, in addition to cell lysis.

### I. Conventional Thermal Shift Assays (C-TSAs)

Protein-ligand interactions are not only involved in almost every process in biological systems, but are also play a role in the external modulation of protein function by drugs. Frederick et al. ,“Conformational entropy in molecular recognition by proteins” Nature 448:325-329 (2007). Protein thermal shift assays (TSAs) are a set of techniques to investigate protein-ligand interactions by detecting the changes in thermodynamic stability of the protein under varying conditions, including ligand binding. Huber et al., “Proteome-wide drug and metabolite interaction mapping by thermal stability profiling” Nat. Methods 12:1055-1057 (2015); and Vedadi et al., “Chemical screening methods to identify ligands that promote protein stability, protein crystallization, and structure determination” Proc. Natl. Acad. Sci. USA 103: 15835-15840 (2006). Multiple TSA's methods have been implemented, however, they have a variety of drawbacks. For example, mass spectrum-based TSAs are very expensive and labor/time intensive while other fluorescence-based TSA approaches suffer from sensitivity issues. These disadvantages limit the capability of these techniques to detect small thermal shifts, particularly for low abundance or large, complex proteins. Other limitations include, but are not limited to, time-consuming workflow, expensive equipment, technical experience, a lack of reliant labels such as antibodies, and/or demand for large amounts of purified proteins. Huynh et al., “Analysis of protein stability and ligand interactions by thermal shift assay” Curr. Protoc. Protein Sci. 79:28-29 (2015); Jafari et al., “The cellular thermal shift assay for evaluating drug target interactions in cells” Nat. Protoc. 9:2100 (2014); and Dart et al., “Homogeneous Assay for Target Engagement Utilizing Bioluminescent Thermal Shift” ACS Med. Chem. Lett. 9:546-551 (2018). These limitations present a barrier to the broader applications of TSA in life science and medicine, particularly in the areas sensitive to efficiency and cost such as pharmaceutical industry and fast diagnosis.

As one of the primary analysis tools of life and chemistry scientists, protein thermal shift assays (also known as thermal denature assay, differential scanning fluorimetry, or ThermoFluor) are an effective assay in measuring purified protein-drug engagement in the drug industry and in detecting protein interaction in academia. Protein thermal stability is very sensitive to protein dynamics and will change accordingly when protein binds to other molecules such as ligand or other protein to form complexes. Such thermal stability change is called thermal shift and can be measured using fluorescence, western blot, or mass spectrometer analysis. Molina et al., “Monitoring drug target engagement in cells and tissues using the cellular thermal shift assay” Science 341:84-87 (2013). Recently a modified thermal shift assay called cellular thermal shift assay (CETSA) can measure intracellular protein thermal shift and provide insight of protein interactions within living cells. Tan Tan, et al., “Thermal proximity coaggregation for system-wide profiling of protein complex dynamics in cells” Science 359:1170-1177 (2018).

### II. Standing Acoustic Wave Technology

Here is disclosed the first thermal shift assay enabled by acoustic mechanisms (an acousto-thermal shift assay (A-TSA)). In one embodiment, surface acoustic waves (SAWs) are employed to unfold proteins and concentrate precipitated proteins on a microfluidic chip by coupling acoustic heating with acoustic forces.

When an acoustic field is imposed on a fluid, it exerts acoustic forces on suspended particles (e.g., proteins or microparticles) induced by acoustic scattering and also on the suspending fluid thereby causing acoustic fluid streaming due to viscous attenuation of the acoustic energy in the fluid. Such acoustic forces have been reported to manipulate fluid, particles, and cells. Ding et al., “Surface acoustic wave microfluidics” Lab on a Chip 13:3626-3649 (2013); Friend et al., “Microscale acoustofluidics: Microfluidics driven via acoustics and ultrasonics” Reviews of Modern Physics 83:647 (2011); Ding et al., “On-chip manipulation of single microparticles, cells, and organisms using surface acoustic waves” Proc. Natl. Acad. Sci. USA 109:11105-11109 (2012); Collins et al., “Two-dimensional single-cell patterning with one cell per well driven by surface acoustic waves” Nat. Commun. 6:8686 (2015); Franke et al., “Surface acoustic wave actuated cell sorting (SAWACS)” Lab on a Chip 10:89-794 (2010); and Frommeltet al., “Microfluidic mixing via acoustically driven chaotic advection. “Physical review letters 100:034502 (2008).

Acoustic heating results from viscous attenuation of the acoustic energy into the fluid and was typically considered in the art as a major hurdle for biomedical applications because the temperature rise is usually not compatible with biological samples. Recent studies have shown that a well-controlled acoustic heating can be a valuable asset in driving chemical and biological reactions. Kulkarni et al., “Surface acoustic waves as an energy source for drop scale synthetic chemistry” Lab on a Chip 9:754-755 (2009); Reboud et al., “Shaping acoustic fields as a toolset for microfluidic manipulations in diagnostic technologies” Proc. Natl. Acad. Sci. USA 109:15162-15167 (2012).; and Shilton et al., “Rapid and controllable digital microfluidic heating by surface acoustic waves” Adv. Funct. Mater. 25;5895-5901 (2015).

In one embodiment, the present invention contemplates a method comprising a standing acoustic wave (SAW) generator, wherein the SAW generator provides acoustic heating energy and acoustic force energy. In one embodiment, the acoustic heating energy provides fast and precisely controlled temperature ramping that unfolds and precipitates proteins without biological damage to the proteins. In one embodiment, the acoustic force energy drives an aggregation of precipitated proteins along the nodes and/or antinodes of the standing acoustic wave field. In one embodiment, the precipitated protein aggregation concomitantly results in a significantly enhanced local concentration and thereby increases a signal amplitude (e.g., increases the signal to noise ratio of the measured detection signal).

For example, one detection signal for monitoring protein unfolding comprises a gray intensity of precipitated proteins measured as a function of SAW time to analyze the thermal stability of proteins. In one embodiment, the A-TSA assay time is less than 2 minutes as compared to. tens of minutes or hours for conventional TSAs. In one embodiment, the A-TSA sensitivity is up to 34-fold higher than conventional TSAs. In one embodiment, the improved sensitivity of the A-TSA can provide detection of small thermal shifts upon protein-ligand bindings to diagnose mutational protein diseases (e.g. sickle cell disease (SCD)).

ps III. Acousto-Thermal Devices

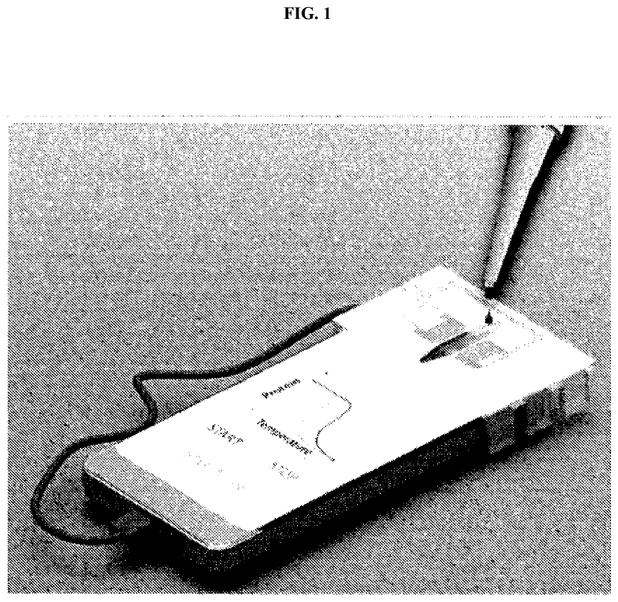

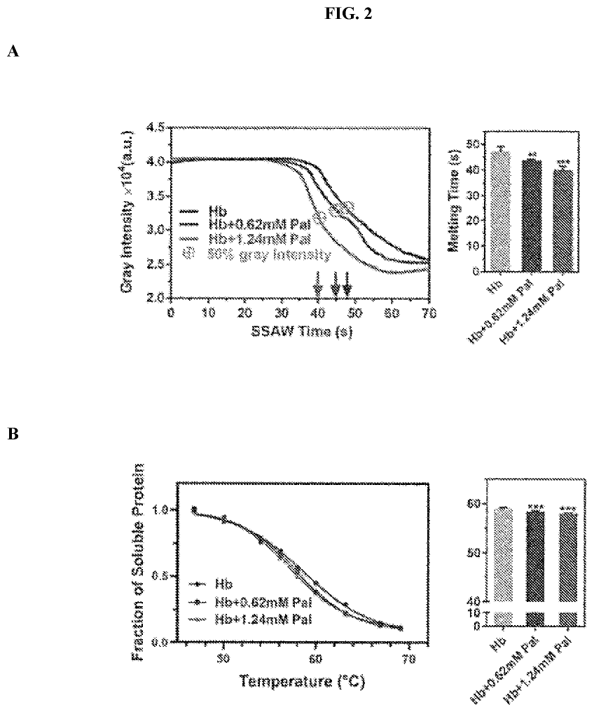

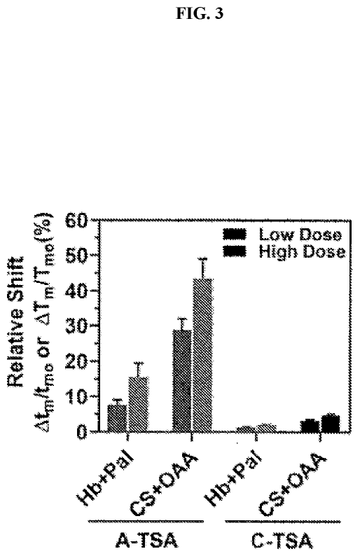

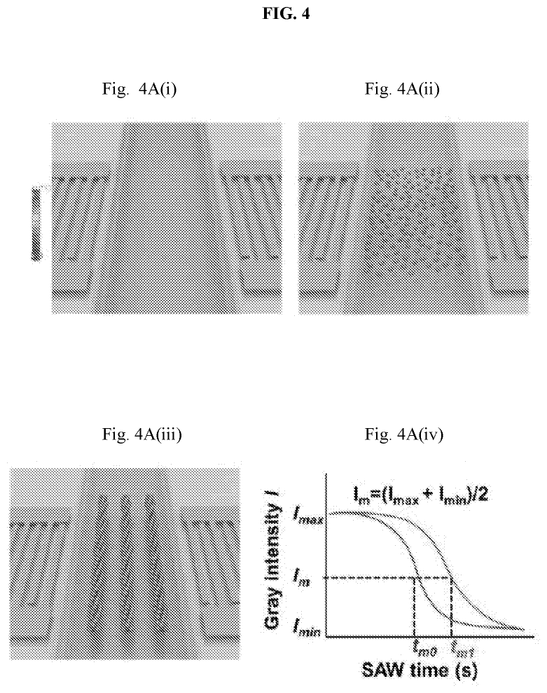

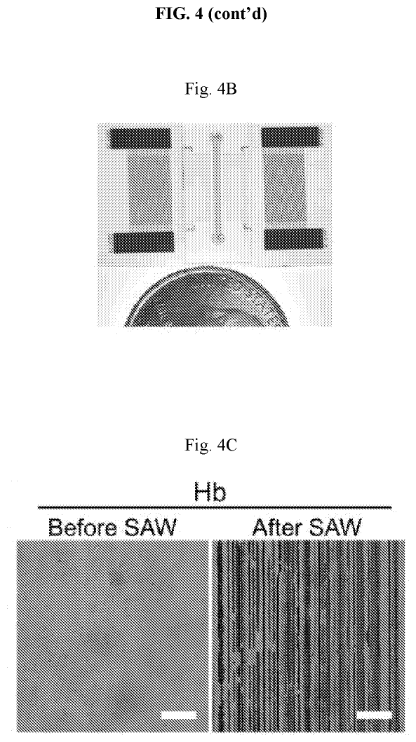

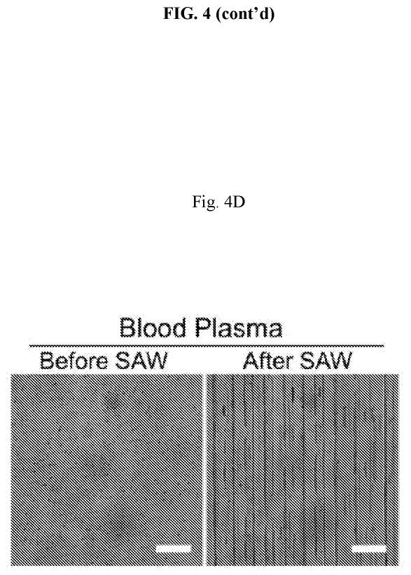

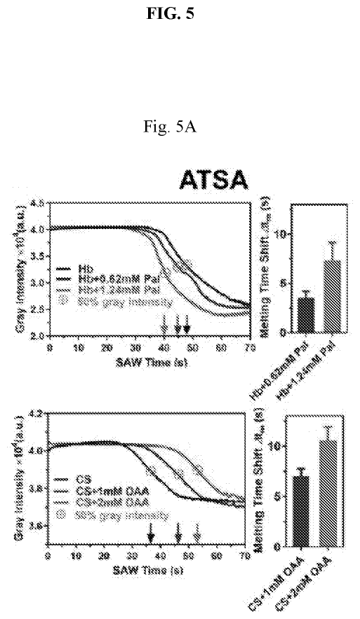

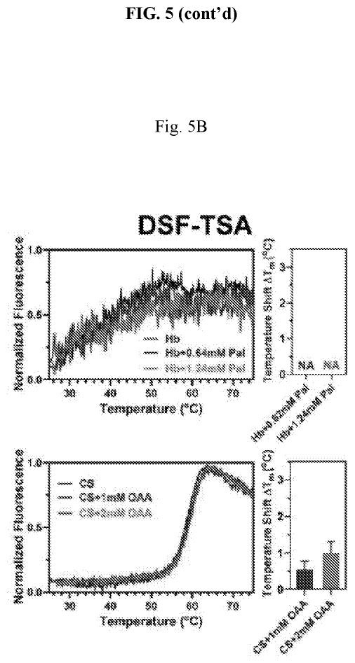

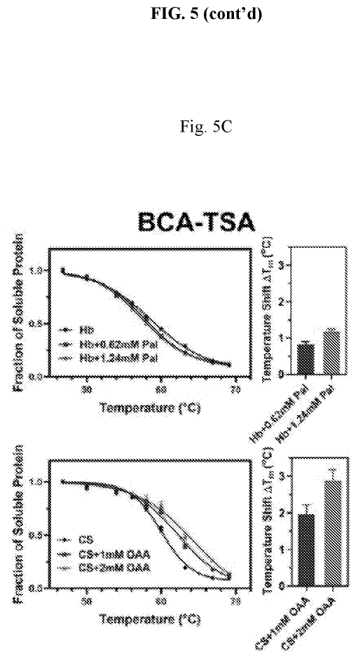

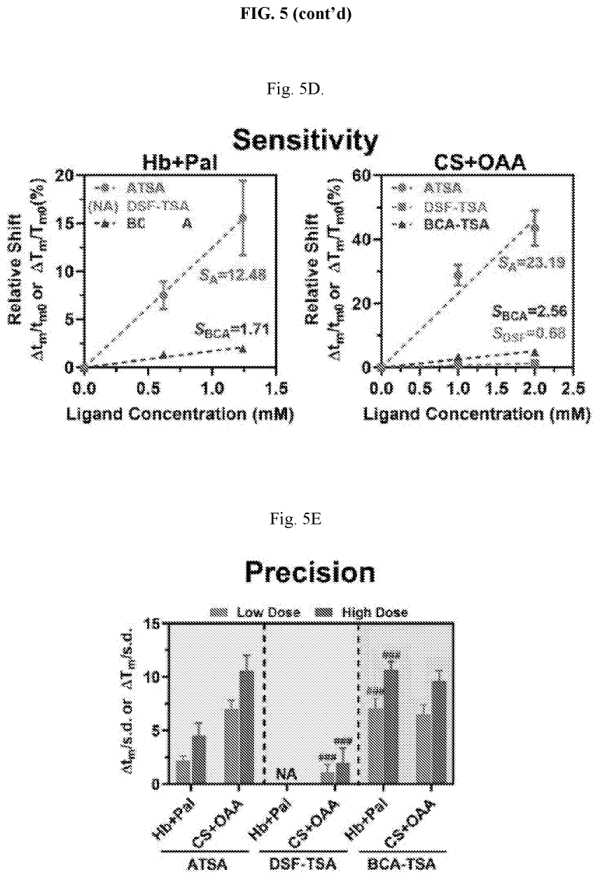

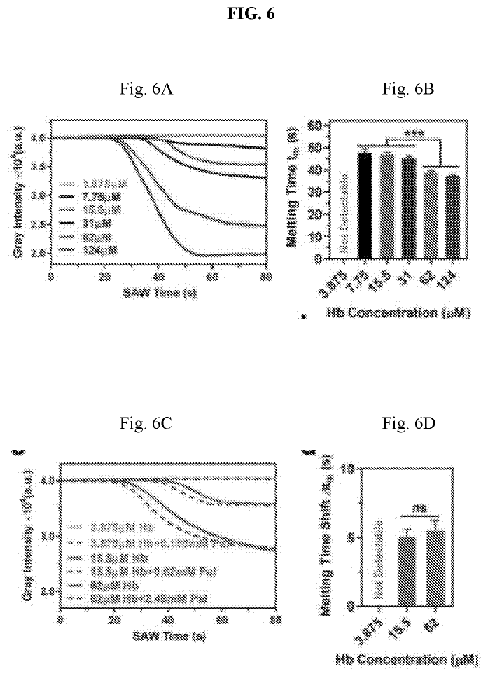

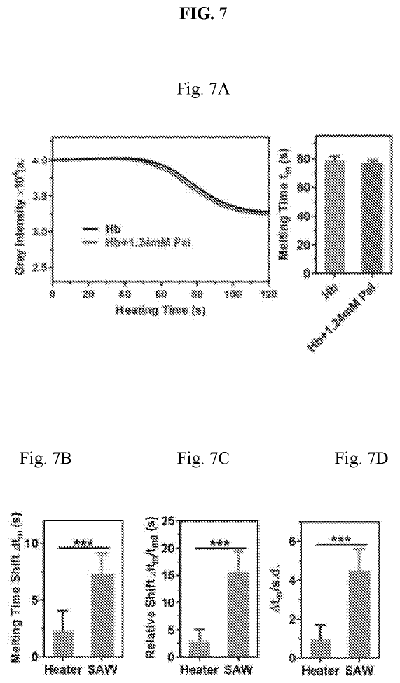

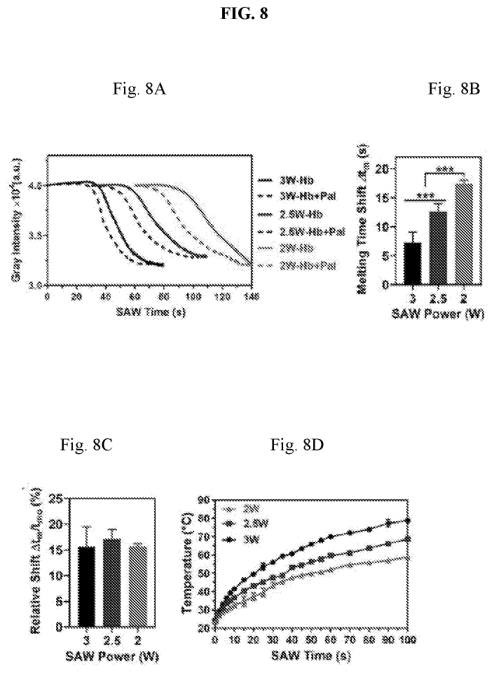

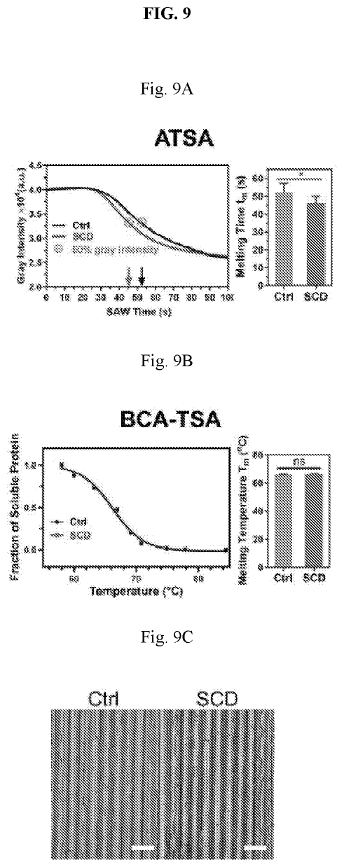

In one embodiment, the present invention contemplates a protein acousto-thermal shift device comprising an acoustic manipulation element and an acoustic heating element. See, FIG. 1. For example, an acousto-thermal assay of the present invention provides a larger melting curve shift at higher concentration of palmatine when bound to hemoglobin as compared to the melting curve obtained by Conventional Thermal Shift Assay (C-TSA:) that measures fluorescence. See, FIG. 2(A) cf (B). Thermal shift curves of palmatine-hemoglobin and citrate synthase-oxaloacetate were obtained showing that the acousto-thermal shift device is much more sensitive than traditional fluorescent thermal shift assay. See, FIG. 3.

Although it is not necessary to understand the mechanism of an invention, it is believed that such an acousto-thermal shift device performs an assay including, but not limited to, the steps of: (1) sample preparation; (2) sample characterization; and (3) data collection. In one embodiment, the device performs sample preparation, characterization and data collection in less than a few minutes. In one embodiment, the sample has a volume of ˜0.0001 mL. In one embodiment, the acuosto-thermal shift device is integrated into a single microchip. In one embodiment, the acuosto-thermal shift microchip device is operated by a cellular phone. In one embodiment, the sample comprises either purified protein or intracellular proteins.

A. A-TSA Device Design And Characterization

In one embodiment, the present A-TSA device comprises an acoustic element that generates surface acoustic waves (SAWs). Surface acoustic waves are believed to be a kind of sound and is a mechanical wave that propagates at the surface of solid substrate. In one embodiment an SAW generator applies an AC signal to a pair of interdigital transducers (IDTs) on top of a piezoelectric material. Although it is not necessary to understand the mechanism of an invention, it is believed that, once generated, an SAW propagates into the interface of a substrate and a liquid media. Further, the SAW may deflect into the liquid media as a leaky Rayleigh wave that can be used as an acoustic tweezer to: i) manipulate and pattern micro/nano particles/molecules (e.g., proteins: ii) generate fluid streaming; and iii) generate acoustic heating.

It has been reported that such acoustic tweezers have many demonstrated applications including: i) on-chip manipulation of C. elegans worms, cells, and nanoparticles; ii) tunable patterning of cells and molecules; iii) separation of label-free cancer cells; iv) cell lysis; and v) acoustic heating. Acoustic fields can produce rotational vortices that mechanically lyse both red blood cells and parasitic cells in a drop of blood as well as streaming at low powers. Although acoustic heating was described, cell lysis was not reported, nor was SAW-patterning of precipitated proteins in a microfluidic chamber. Reboud, J., et al., “Shaping acoustic fields as a toolset for microfluidic manipulations in diagnostic technologies” PNAS USA 109(38):15162-15167, (2012). Mild sound wave intensities and frequencies have been disclosed to process cells in a manner that did not damage biological cells. Consequently, it is known that SAW preserves cell integrity during an acoustic separation process. While it is demonstrated that small radio frequency power can support a fully integrated cell separation and analysis system surface acoustic wave cell lysis is not shown. Ding, X., et al., “Cell separation using tilted-angle standing surface acoustic waves” PNAS USA 111:12992-12997 (2014). A noninvasive acoustic based method has been reported that manipulates single microparticles, cells or entire organisms in a microfluidic chip utilizing surface resonance to gently manipulate the sample. Cell viability tests were performed to verify the technologies compatibility with biological objects, but cell lysis with SAW resonance is not shown. Ding, X., et al., “On-Chip Manipulation of Single Microparticles, Cells, and Organisms Using Surface Acoustic Waves” PNAS USA 109:11105-11109 (2012). Acoustic radiation force providing a continuous flow of tunable surface acoustic waves has been reported to directly manipulate and sort cells, rather than using acoustic streaming to manipulate the fluids in which the cells are embedded but cell lysis and releasing intercellular proteins with surface acoustic waves was not shown. Ding, X., et al., “Surface acoustic wave (SAW) microfluidics” Lab on a Chip 13:3626-3649 (2012). The prior art fails to disclose that SAW results in protein precipitate and protein patterning.

However, a combination of multiple acoustic functions and effects into one device for protein thermal shift analysis has not been reported yet. Because of the great controllability of SAW, the present invention contemplates an acoustic thermal effect that precisely controls the temperature of a sample over a region of interest within a microfluidic channel or chamber. If proteins are present, a thermal induced protein unfolding results in the formation of a protein precipitate. Simultaneously, a standing SAW is formed that induces a pattern in the precipitated proteins taking the form of parallel lines or arrays between the acoustic pressure nodes or antinodes. Although it is not necessary to understand the mechanism of an invention, it is believed that such protein precipitate patterns dramatically enhances the local concentration of proteins, thus increasing measurement sensitivity.

Protein gray intensity can be measured by a camera (e.g., a cellular phone camera) at a series of time points through the whole precipitation process (about 10-50 seconds) to measure the melting curve and melting temperature. The melting temperature or melting curve shift provides immediate data regarding protein dynamics, interaction, or configuration. It is further belied that there is: i) a relationship between the gray density of protein precipitate and its concentration; ii) a protein response to acoustic waves over a broad range of frequency and amplitude; iii) an A-TSA application for diagnosis of diseases that is related to protein disorders (e.g., hemoglobinopathies) where modified proteins have melting temperature shifts; iv) an interaction between acoustic waves and protein dynamics that affects protein thermal stability in A-TSA.

Specifically, two identical SAWs were generated by applying an AC (alternating current) signal to a pair of interdigital transducers (IDTs) deposited on the surface of a lithium niobate piezoelectric substrate. A standing SAW was formed within a 1×10 mm² polydimethylsiloxane (PDMS) microchannel. The microchannel was bonded on top of a substrate between these two IDTs. See, FIGS. 4A(i) and FIG. 4B. Under SAW actuation (i.e., 19.6 MHz, 3 Watt), the temperature of a small-volume protein solution (e.g., less than 2 μL) in phosphate-buffered saline (PBS) within the microfluidic channel can be rapidly increased from 23° C. to 80° C. within 100 s. Most proteins rapidly precipitate and aggregate after their unfolding. See, FIG. 4A(ii).

Simultaneously, the standing SAW aggregates and concentrates precipitated protein along the acoustic pressure nodes and/or antinodes. See, FIG. 4A(iii). Gray intensity (Iₘ) of the precipitated and aggregated proteins was analyzed and plotted as a function of SAW time, giving rise to a sigmoidal melting curve. See, FIG. 4A(iv). The melting time (Tₘ) was determined as the time point when half of proteins were unfolded (i.e. herein, a 50% change of gray intensity occurs where Iₘ=(Iₘₐₓ+Iₘᵢₙ)/2.). Using this simple analysis, the time shift Δτₘ between between two samples can be measured at a given SAW power and analyzed to compare conditions or ligands that stabilize or destabilize proteins. Overall, this new technique provides a rapid, simple and efficient workflow for analysis of changes in apparent melting curves.

### IV. Acousto-Thermal Assay Methods

In one embodiment, the present invention contemplates a method for protein analysis, comprising: a) providing an acousto-thermal shift device comprising a microchannel and an acoustic element; b) loading a small amount of sample is loaded into the microchannel; c) heating the sample with said acoustic element to a first precise temperature; d) streaming and mixing the sample with said acoustic element, wherein cells in the sample are lysed and release intracellular proteins; and e) manipulating the intracellular proteins into specific patterns with said acoustic element under a second precise temperature, wherein the specific patterns are parallel lines or arrays within the microchannel which significantly enhance the local protein concentration and achieve a very high signal-noise ratio. In one embodiment, the acousto-thermal shift device performs steps a)-e) on a single microchip within a few minutes from loading sample to reading out the measured data.

Although it is not necessary to understand the mechanism of an invention, it is believed that low sample volumes and short assay times has not been previously reported for thermal shift assay devices. It is further believed that such advantages have great impact across multiple disciplines including, but not limited to: (1) drug industries by dramatically reducing the time cost for drug screening and drug discovery; (2) clinical applications by providing a low cost, fast, portable, and label-free tool for proteopathy diseases (protein disorder diseases) diagnosis such as Alzheimer's disease, prion diseases, amyloid, sickle disease etc., and for real-time monitoring about the efficacy of therapies; and (3) academia by providing a low cost and fast protein analysis device.

Most proteins are relatively soluble in aqueous solution at room temperature and can be denatured and/or unfolded at a certain temperature to form a precipitate. Each protein has a precise denaturation temperature that is generally known in the art as a melting temperature (Tₘ). Variations in melting temperature is very sensitive to protein configuration and its interaction with a binding ligand or other proteins. Consequently, a protein melting temperature shifts when the protein binds to a ligand or other proteins to form a complex. As a result, the observed melting temperature shift upon formation of these complexes can be used to study and determine protein dynamics, interactions, or status. In one embodiment, the present invention contemplates an acousto thermal shift device and assay (A-TSA) that utilizes surface acoustic wave-induced: i) acoustic heating; ii) acoustic streaming; and iii) acoustic patterning/manipulation to integrate cell lysis, heating, protein concentration, and data measurement all in one single step within one single microchip.

The data presented herein demonstrate protein unfolding and concentration of a purified protein of human hemoglobin (Hb) under SAW actuation. See, FIG. 4C. Hb is the most abundant protein in red blood cells (RBC) and is dependent upon interactions with other molecules in blood for its functions. Nagy et al., “Red cells, hemoglobin, heme, iron, and atherogenesis. Atertio. Thromb. Vasc. Biol. 30:1347-1353 (2010). Protein solutions in the microfluidic channel are usually visually clear before SAW actuation. When SAW actuation (19.6 MHz, 3 Watt) commences, the acoustic heating causes the proteins to unfold and precipitate. With continued SAW exposure, the precipitated proteins quickly aggregate along nearby pressure nodes and anti-nodes under acoustic forces to form concentrated protein microfibers. See, FIG. 4C. Similar SAW-driven protein unfolding and concentration were also observed with a mixed protein solution of human blood plasma. See, FIG. 4D. Together, these data show that SAW exposure results in rapid protein unfolding, precipitation, and concentration, which provides for a protein thermal stability analysis.

A. Assessment of Protein-Ligand Binding

In some embodiments, SAW-driven protein unfolding and concentration can be used for analysis of protein-ligand binding to analyze the melting temperature (Tₘ) and its shift (Δtₘ) upon protein-ligand binding. Gray intensity of the precipitated and concentrated proteins was measured and plotted as a function of SAW time in A-TSA. See, FIG. 5A. Two control conventional TSA methods were performed in parallel measuring the data using either a differential scanning fluorimetry (DSF) assay or a bicinchoninic acid (BCA) assay. DSF, the most popular C-TSA method, utilizes dye fluorescence as a measure of protein unfolding. Matulis et al., “Thermodynamic stability of carbonic anhydrase: measurements of binding affinity and stoichiometry using ThermoFluor” J. Biochemistry 44:5258-5266 (2005). On the other hand, BCA quantifies the amount of remaining soluble proteins after thermal unfolding. Brown et al., “Protein measurement using bicinchoninic acid: elimination of interfering substances” Anal. Biochem. 180:136-139 (1989).

Two compounds, palmatine chloride (Pal) and oxaloacetic acid (OAA), were selected due to interact with (e.g., bind to) Hb and CS, respectively. Liu et al., “Studies on the interaction of palmatine hydrochloride with bovine hemoglobin” Luminescence 29:211-218 (2014); and Niesen et al., “The use of differential scanning fluorimetry to detect ligand interactions that promote protein stability” Nat. Protoc. 2:2212 (2007). Under SAW actuation (e.g., 19.6 MHz, 3 Watts), a typical sigmoidal melting curves of Hb and Hb-Pal complexes reveals that the Tₘ of Hb (46.85 s) was reduced by 3.52 s and 7.30 s in the presence of 0.62 mM and 1.24 mM Pal, indicating destabilization of Hb by Pal. See, FIG. 5A.

Exemplifying the superiority of the presently disclosed A-TSA, the data shows that DSF-TSA could not successfully produce such typical sigmoidal melting curves for Hb and Hb-Pal complexes due to the inference from the intrinsic fluorescence of both Hb and Pal. See, FIG. 5B; and Alpert et al., “Tryptophan emission from human hemoglobin and its isolated subunits” Photochem. Photobiol. 31:1-4 (1980). Alternatively, another conventional TSA method, BCA assay, was performed to analyze at tₘ shift of Hb against Pal. The results showed that the Tₘ of Hb (59.1° C.) was decreased by 0.7° C. and 1.1° C. in the presence of 0.62 mM and 1.24 mM Pal. See, FIG. 5C. Under the same SAW actuation, the tₘ shift of CS (36.23 s) was increased by 10.45 s and 15.77 s in the presence of 1 mM and 2 mM OAA, due to their known stabilizing effect on CS. By contrast, the tₘ shift of CS as detected in DSF assay was small and not statistically significant although the typical sigmoidal melting curves were obtainable. See, FIG. 5B.

Likewise, the Tₘ of CS (60.3° C.) was increased by only 2.0° C. and 2.9° C. in the presence of 1 mM and 2 mM OAA as detected in the BCA assay. See, FIG. 5C. In addition, the analysis of thermal stability of Hb and Hb-Pal complex with various concentrations showed that the detected tₘ and Δtₘ were not visibly sensitive to concentrations varying from 124 μM to 3.875 μM in current ATSA. See, FIGS. 6A-D.

To make a direct comparison between the presently disclosed A-TSA and C-TSA methods, their relative sensitivity was evaluated by defining the relative shift (Δtₘ/tₘ₀ or ΔTₘ/Tₘ₀) and sensitivity (tangential slope). The results showed that the presently disclosed A-TSA showed superior and enhanced sensitivity in monitoring the protein-ligand binding compared to the C-TSAs DSF-TSA and BCA-TSA. See, FIG. 4D. Sensitivity was also shown to be dependent on the protein type and compound concentration. Specifically, the presently disclosed A-TSA achieved a sensitivity of 7-fold higher in Hb-Pal binding and 9-fold higher in CS-OAA binding than BCA-TSA. More strikingly, the presently disclosed A-TSA method produced a sensitivity of 34-fold higher than DSF-TSA method for CS-OAA binding.

Precision, as defined by the ratio between Δtₘ (or ΔTₘ) and standard deviation (s.d.) of tₘ (or Tₘ), i.e. Δtₘ s.d. (or ΔTₘ s.d.), was evaluated. See, FIG. 4E. The presently disclosed A-TSA demonstrated a much higher precision than DSF-TSA method while comparable to BCA-TSA method. The data also showed that sensitivity and precision were significantly compromised when protein unfolding was induced under a heating-only condition instead of under SAW induced heating and concentration, suggesting that acoustic-mediated protein concentration is specific for A-TSA. See, FIG. 7A-D.

In addition, the present data shows that the magnitude of melting time shift Δtₘ in A-TSA could be facilely tuned by varying the SAW power. See, FIG. 8. The magnitude of Δtₘ between Hb and Hb-Pal complex were significantly increased and the Δtₘ became more viable by lowering the SAW power from 3 W to 2.5 W or 2 W. See, FIGS. 8A and 8B. Although, the relative shifts seemed not to be visibly sensitive to SAW power. See, FIG. 8C. This tunability was attributed to the slower heating profiles under lower SAW power. See, FIG. 8D. Lower SAW power would benefit the detection of marginal thermal shifts upon ligand bindings that might not be distinctly revealed by C-TSAs.

B. Assessment of Protein Mutation

These promising results encouraged us to further investigate the potential of our ATSA method for diagnosis of mutational protein diseases, which are associated with protein misfolding and subsequent thermal stability changes. Cohen et al., “Therapeutic approaches to protein-misfolding diseases” Nature 426:905-909 (2003). Sickle cell disease (SCD) is one example which affects millions worldwide and is caused by polymerization of sickle Hb in individual RBCs. Piel et al., “Sickle cell disease” New Engl. J. Med. 376:1561-1573 (2017). However, the lack of practical diagnostic approach leads to an inability to early treatment and high childhood mortality especially in resource-limited areas. Ilyas et al., “Emerging Point-of-Care Technologies for Sickle Cell Disease Diagnostics” Clin. Chim. Acta (2019). Thermodynamic instability of sickle Hb is reported to be characteristic of SCD. Tam et al., “Sickle cell hemoglobin with mutation at αHis-50 has improved solubility. J. Biol. Chem. 290:21762-21772 (2015); and Meng et al., “Substitutions in the β subunits of sickle-cell hemoglobin improve oxidative stability and increase the delay time of sickle-cell fiber formation” J. Biol. Chem. 294:4145-4159 (2019).

We examined whether our technique could distinguish the Hb stability profiles with red blood cell (RBC) lysate from healthy (Ctrl) and SCD human donors. See, FIG. 9. (FIG. 4). The melting time tm of RBC lysates from healthy and SCD donors were 52.13 s and 45.98 s respectively, producing an apparent shift of Δtm in our ATSA method. See, FIG. 9A. (FIG. 4a). By contrast, the melting curves produced by the conventional BCA-TSA method were almost overlapped and no significant melting temperature shift ΔTm was detected between healthy and SCD RBC lysates, indicating its inefficacy to distinguish them. See, FIG. 9B (FIG. 4b).

In addition, under SAW actuation, the parallel-aligned protein patterns were formed in the microfluidic channels and no visible difference in their morphology was observed between healthy and SCD Hb. See, FIG. 9C. (FIG. 4c). Our technique demonstrates here a potential application as a new and promising point-of-care platform for rapid and highly sensitive diagnostic tool of SCD, although the further optimization is required for clinical use.

### Experimental

### EXAMPLE I

### Chemicals

Two proteins, i.e. human hemoglobin (Hb; Millipore Sigma, St. Louis, Mo., USA) and citrate synthase (CS) from porcine heart (Millipore Sigma), and two corresponding compounds, i.e. palmatine chloride (Pal; Santa Cruz Biotechnology, Santa Cruz, Calif., USA) and oxaloacetic acid (OAA; Millipore Sigma) were primarily used. Human whole blood samples with K₂EDTA as an anticoagulant were purchased from Zen-Bio Inc. (Research Triangle Park, N.C., USA) and stored in 4° C. (always used within 3-14 days after collection). Human blood plasma was obtained by centrifuging the human whole blood samples at 500 g for 10 min in 4° C. Sickle cell diseased (SCD) red blood cell lysate were obtained from patients with SCD, upon receiving written informed consent and in conformity with the declaration of Helsinki under a protocol approved by the Duke University Medical Center (no. NCT02731157) as described previously. Culp-Hill et al., “Effects of red blood cell (RBC) transfusion on sickle cell disease recipient plasma and RBC metabolism” Transfusion 58:2797-2806 (2018). All abovementioned chemicals were dissolved or diluted using Dulbecco's Phosphate-Buffered Saline (DPBS; Thermal Fisher Scientific, Hampton, N.H., USA) except Pal that was first dissolved in dimethyl sulfoxide (DMSO; Millipore Sigma) but then further diluted in DPBS. The DMSO concentration is 2% (vol/vol) in final Hb-Pal complex solution in order to prevent the potential damage to the protein by contact with high concentrations of DMSO. Fluorescent microspheres (polystyrene, 7.5 μm diameter) were obtained from Bangs Laboratories Inc. (Fishers, Ind., USA).

### EXAMPLE II

### A-TSA Device Fabrication

The SAW was generated and propagated on piezoelectric 128° Y-cut X-propagating lithium niobate (LiNbO₃) wafer (500 μm thick). The device consisted of a pair of interdigitated transducers (IDTs) in parallel in order to generate two series of identical SAWs propagating in the opposite direction, producing a standing SAW. Each IDT consists of 30 pairs of electrodes (Cr/Au, 5/100 nm) with the width of electrode finger of 50 μm, pitch of 100 μm, and an aperture of 10 mm, yielding a frequency of approximately 20 MHz for the propagating SAW. Although different IDTs can be used, the resonance frequencies of most IDTs are in the range between 19.5 and 19.6 MHz. A PDMS microchannel with height of 100 μm and width of 2 mm was then fabricated through a standard soft-lithography and model-replica procedure. Lastly, both the PDMS channel and the IDT substrate were treated with oxygen plasma and bonded together to form the final SAW device. See, FIG. 4B.

### EXAMPLE III

### Acousto-Thermal Shift Assay (A-TSA) and Data Analysis

The A-TSA SAW device was mounted on the stage of an inverted microscope (ECLIPSE Ti-U, Nikon, Japan). A radio frequency (RF) signal was generated by a function generator (EXG Analog Signal Generator, Keysight, Santa Rosa, Calif., USA) and amplified by an amplifier (403LA, Electronics & Innovation, Rochester, N.Y., USA). Five microliters of protein, plasma, red blood cell lysate or protein-compound mix solutions were injected into the channel before the RF signals were applied. A fast camera (ORCA-Flash4.0LT, Hamamatsu, Japan) was connected to the microscope to capture the process, and all the videos were recorded in 4 frames per second.

All image and videos processing were performed in ImageJ (National Institute of Health, Bethesda, Md., USA) in the same way as described below. The same sized regions of interest (ROIs) were traced around the perimeter of each pattered protein fiber in order to monitor the gray intensity and its change during the course of protein melting and aggregation. At least five ROIs were selected and characterized for each video. Melting time was defined as the time point when there is 50% gray intensity change Iₘ=(Iₘₐₓ+Iₘᵢₙ)/2.

### EXAMPLE IV

### Conventional Thermal Shift Assay

Two conventional methods were adopted for thermal shift assay: i) SYPRO differential scanning fluorimetry (DSF) assay; and ii) bicinchoninic acid (BCA) assay.

SYPRO DSF assay: SYPRO Orange melting curves were collected using the 7900HT Fast Real-Time PCR System. The SYPRO Orange fluorescent signal is detectable using the calibration setting for the ROX filter. Melting curves were performed using 1 mg/mL of protein with a 1:2500 dilution of SYPRO Orange (Molecular Probes Inc #S-6651) in 100 mM PBS, pH 7.4, using a minimum of 4 replicates. A 1% ramp rate from 25° C. to 95° C. was utilized during data collection. Drug concentrations are as indicated. To analyze melting curves, the fluorescence was normalized to the starting temperature and to no protein controls. The data was then scaled to interval (0,1) and then the replicates were averaged, and standard deviation calculated.

BCA assay: For thermal gradient profiling, a gradient program was created using a PTC-200 thermal cycler (MJ Research, Reno, N.V., USA) to cover the temperature points indicated in each figure. A PCR plate was prepared with 25 μL per well of recombinant protein or lysate and sealed (4titude Random Access, PN 4ti-0960/RA 96-well plate). The plates were spun at 1200 g for two minutes at 4° C., and then kept at 4° C. prior to use. The plate was placed in the thermal cycler with the heated lid closed for 3 minutes and was then spun at 1200 g for two minutes to remove any condensation. The PCR tubes were removed from the PCR plate, carefully placed in 1.5 mL tubes, and spun at 21,000 g for 30 min at 4° C. to pellet the aggregate protein. Supernatant was carefully removed from each tube and placed in a clean, low-retention, 1.5 mL tubes. 10 ul of solution was removed and a Pierce BCA protein assay kit (PN 23225) was used for the determination of the total protein in each sample.

### EXAMPLE V

### Statistical Analysis

All data were expressed as means±SD. The data (melting time and melting temperature) from multiple runs (n≥3) were plotted using Graphpad Prism 8.0 software (GraphPad Software Inc., La Jolla, Calif., USA). The BCA-TSA data were fitted using a Sigmoidal dose-response (variable slope) curve fit. Unpaired t-test or ordinary one-way ANOVA with Tukey's multiple comparison test was used to analyze statistical significance. A p-value<0.05 was considered statistically significant. Within the figures, the significance was denoted by the following marks: * or # for p<0.05; ** or ## for p<0.01; and *** or ### p<0.001.

We claim:

1. A method, comprising:
a) providing:
i) an acousto-thermal device comprising a surface acoustic wave source and at least one microfluidic channel or chamber; and
ii) a sample comprising at least one protein;
b) introducing said sample into said at least one microfluidic channel or chamber;
c) controlling the temperature of the said sample with said surface acoustic wave source to a plurality of precise temperatures within said at least one microfluidic channel or chamber under conditions that create a precipitated protein; and
d) aggregating said precipitated protein with said surface acoustic wave into a pattern.

2. The method of claim 1, wherein said pattern comprises parallel lines or arrays.

3. The method of claim 1, wherein said aggregating increases a local concentration of said precipitated protein.

4. The method of claim 1, wherein said aggregating is performed simultaneously with said protein precipitation.

5. The method of claim 1, further comprising measuring protein gray intensity.

6. The method of claim 5, wherein said protein gray intensity measurements determine a protein melting curve.

7. The method of claim 1, wherein said sample comprises plurality of biological cells.

8. The method of claim 7, further comprising lysing at least a portion of said plurality of biological cells with said surface acoustic wave source.

9. An acousto-thermal device, comprising:
i) a piezoelectric substrate comprising at least one microchannel or chamber;
ii) at least two parallel interdigital transducers deposited longitudinally in said at least one microchannel or chamber; and
iii) a fluid comprising a plurality of proteins disposed between said at least two parallel interdigital transducers.

10. The device of claim 9, wherein each of said parallel interdigital transducers comprises thirty (30) pairs of electrodes.

11. The device of claim 9, wherein each of said electrode pairs comprise chromium and gold.

12. The device of claim 9, wherein each of said electrode pairs have a thickness of approximately 5/100 nm.

13. The device of claim 9, wherein each of said electrode pairs comprise an electrode finger of 50 μm in length, a pitch of 100 μm, and an aperture of 10 mm.

14. The device of claim 9, wherein each of said electrode pairs yield a standing acoustic wave having a frequency of approximately 20 MHz.

15. The device of claim 9, wherein said piezoelectric substrate comprises a material selected from the group consisting of silicon, glass, plastic, quartz and polydimethylsiloxane (PDMS).

16. A method, comprising:
a) providing:
i) an acousto-thermal device comprising a surface acoustic wave source and at least two microfluidic channels or chambers;
ii) a first sample comprising at least one first protein disposed in a first microfluidic channel or chamber; and
iii) a second sample comprising at least one second protein disposed in a second microfluidic channel or chamber;
b) controlling the temperature of said first and second sample with said surface acoustic wave source to a plurality of precise temperatures within said microfluidic channel or chamber under conditions that create a first and second precipitated protein;
c) aggregating said first and second precipitated protein with said surface acoustic wave into a first and second pattern;
d) measuring a gray intensity of said first and second precipitated protein;
e) determining a first and second melting temperature of said first and second precipitated protein; and
f) calculating a difference between said first and second melting temperature with a three to thirty-five-fold increased sensitivity as compared to conventional thermal shift assays.

17. The method of clam 16, wherein said second protein is bound to a ligand.

18. The method of claim 17, wherein said ligand is selected from the group consisting of a small organic molecule, an antibody and a protein.

19. The method of claim 16, wherein said second protein comprises a mutation as compared to a wild type sequence.

20. The method of claim 16, said difference diagnoses a genetic disease.

21. The method of claim 16, wherein said pattern comprises parallel lines or arrays.

22. The method of claim 16, wherein said aggregating increases a local concentration of said precipitated protein.

23. The method of claim 16, wherein said aggregating is performed simultaneously with said protein precipitation.

24. The method of claim 16, wherein said sample comprises plurality of biological cells.

25. The method of claim 16, wherein said method further comprises lysing at least a portion of said plurality of biological cells with said surface acoustic wave source.
